(19) United States
(12) Patent Application Publication
(10) Pub. No.: US 2004/0115155 A1
(43) Pub. Date: Jun. 17, 2004

(54) Hair conditioning composition comprising cellulose polymer

(75) Inventors: Dorothy Yong Juanico Salvador, Ashiya Hyogo (JP); Ananthanarayan Venkateswaran, Higashinada-ku (JP); Chisato Anada, Higashinada-ku (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/730,437

(22) Filed: Dec. 8, 2003

Related U.S. Application Data
(63) Continuation of application No. PCT/US01/18745, filed on Jun. 8, 2001.

(51) Int. Cl. A61K 7/06; A61K 7/11

(52) U.S. Cl. 424/70.13

(57) ABSTRACT

Disclosed is a hair conditioning composition comprising by weight:

(a) from about 0.001% to about 5% of a cellulose polymer having a molecular weight of from about 10,000 to about 10,000,000;

(b) from about 0.01% to about 10% of a cationic surfactant;

(c) from about 0.01% to about 15% of a high melting point fatty compound having a melting point of 25° C. or higher; and

(d) an aqueous carrier.

### CROSS REFERENCE TO RELATED APPLICATION

The application is a continuation of International application PCT/US01/18745 (Case AA553F) filed on Jun. 8, 2001.

### TECHNICAL FIELD

The present invention relates to a hair conditioning composition containing a cellulose polymer.

### BACKGROUND

Human hair becomes soiled due to its contact with the surrounding environment and from the sebum secreted by the scalp. The soiling of hair causes it to have a dirty feel and an unattractive appearance. The soiling of the hair necessitates shampooing with frequent regularity.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils and other natural conditioning and moisturizing components. The hair can further be left with increased levels of static upon drying, which can interfere with combing and result in a condition commonly referred to as “fly-away hair”, or contribute to an undesirable phenomena of “split ends”, particularly for long hair.

A variety of approaches have been developed to condition the hair. These approaches range from post-shampoo application of hair conditioners such as leave-on and rinse-off products, to hair conditioning shampoos which attempt to both clean and condition the hair from a single product.

Although some consumers prefer the ease and convenience of a shampoo which includes conditioners, a substantial proportion of consumers prefer the more conventional conditioner formulations which are applied to the hair as a separate step from shampooing, usually subsequent to shampooing. Conditioning formulations can be in the form of rinse-off products or leave-on products, and can be in the form of an emulsion, cream, gel, spray, and mousse. Such consumers who prefer the conventional conditioner formulations value the relatively higher conditioning effect, or convenience of changing the amount of conditioning depending on the condition of hair or amount of hair.

A common method of providing conditioning benefit to the hair is through the use of hair conditioning agents such as cationic surfactants and polymers, high melting point fatty compounds, low melting point oils, silicone compounds, and mixtures thereof. Most of these conditioning agents are known to provide various conditioning benefits such as moisturized feel, softness, and static control to the hair.

Hair conditioning compositions comprising the conditioning agent described above, can provide conditioning benefits such as softness and moisturized feel on dry hair, however, also weigh down the hair. The weighed down hair gives an appearance of reduced bulk hair volume. For consumers who desire hair volume-up such as consumers having fine hair, the effect of hair weighing down is not desirable. The term “hair volume-up” as used herein is not equal to fly-away hair. Fly-away hair is due to the increased level of static, and represents volume increase of only very minor amount of the hair as a whole, and is not desirable. On the other hand, hair volume-up as used herein relates to increase of the bulk of the hair volume. Consumers having fine hair have the desire to achieve hair volume-up while controlling undesirable fly-away of the hair.

On the other hand, consumers whose hair are easy to expand, such as consumers having coarse, wavy, curly or much volume of hair, desire hair conditioning compositions which provide improved moisturizing benefit to achieve hair volume down as well as other conditioning benefit. The term “hair volume down” as used herein relates to decrease of bulk hair volume, for example, relates to reduced hair expansion.

Based on the foregoing, there remains a desire to provide hair conditioning compositions which provide hair volume control benefit such as hair volume up and hair volume down, while providing conditioning benefits such as softness, moisturized feel, and fly-away control.

None of the existing art provides all of the advantages and benefits of the present invention.

### SUMMARY

The present invention is directed to a hair conditioning composition comprising by weight:

(a) from about 0.001% to about 5% of a cellulose polymer having a molecular weight of from about 10,000 to about 10,000,000;

(b) from about 0.01% to about 10% of a cationic surfactant;

(c) from about 0.01% to about 15% of a high melting point fatty compound having a melting point of 25° C. or higher; and

(d) an aqueous carrier.

These and other features, aspects, and advantages of the present invention will become evident to those skilled in the art from a reading of the present disclosure.

### DETAILED DESCRIPTION

While the specification concludes with claims particularly pointing and distinctly claiming the invention, it is believed the present invention will be better understood from the following description.

All percentages are by weight of the total composition unless otherwise indicated. All ratios are weight ratios unless otherwise indicated. All percentages, ratios, and levels of ingredients referred to herein are based on the actual amount of the ingredient, and do not include solvents, fillers, or other materials with which the ingredient may be combined as commercially available products, unless otherwise indicated.

As used herein, “comprising” means that other steps and other ingredients which do not affect the end result can be added. This term encompasses the terms “consisting of” and “consisting essentially of”.

All cited references are incorporated herein by reference in their entireties. Citation of any reference is not an admission regarding any determination as to its availability as prior art to the claimed invention.

COMPOSITIONS

The hair conditioning composition of the present invention comprises:

(a) from about 0.001% to about 5% of a cellulose polymer having a molecular weight of from about 10,000 to about 10,000,000;

(b) from about 0.01% to about 10% of a cationic surfactant;

(c) from about 0.01% to about 15% of a high melting point fatty compound having a melting point of 25° C. or higher; and

(d) an aqueous carrier.

The composition of the present invention can provide hair volume control benefit such as hair volume up and hair volume down, while providing conditioning benefits such as softness, moisturized feel, and fly-away control.

In one preferred embodiment of the present invention, the composition_(Composition A) comprises by weight:

(a) from about 0.001% to about 5% of the cellulose polymer, preferably the cellulose polymer selected from the group consisting of a hydrophobically modified cationic cellulose; a hydrophobically modified cellulose ether; a copolymer of hydrophilic-cellulose units and diallyldimethyl ammonium chloride units; Polyquaternium-10; guar derivatives; and mixtures thereof;

(b) from about 0.01% to about 10% of a cationic surfactant comprising: an amidoamine and an acid; and a dialkyl dimethyl ammonium salt;

(c) from about 0.01% to about 15% of a high melting point fatty compound having a melting point of 25° C. or higher; and

(d) an aqueous carrier.

Preferably composition A further comprises a polypropylene glycol. Composition A may further contain a silicone compound. Composition A can provide hair volume control benefit such as hair volume up and hair volume down, while providing other conditioning benefits. Furthermore, composition A can provide a translucent composition. Translucent appearance of a conditioner may be aesthetically favorable to the consumers, further, such appearance is suitable for adding various aesthetic materials such as mica and titanium dioxide to further enhance aesthetics.

For providing hair volume up benefit, (i) the combination of an amidoamine and an acid is included in the composition at a level of, preferably from about 0.6% to about 1.8%, more preferably from about 0.9% to about 1.6%, by weight of the composition; and (ii) a dialkyl dimethyl ammonium salt is included in the composition at a level of, preferably from about 0.1% to about 1.5%, more preferably from about 0.25% to about 1.25%, by weight of the composition.

When (i) the combination of an amidoamine and an acid are included in the composition at a level of, preferably from about 1.3% to about 3.3%, more preferably from about 1.6% to about 3.0%, by weight of the composition; and (ii) a dialkyl dimethyl ammonium salt is included at a level of from about 1.5% to about 5.0%, preferably from about 1.5% to about 4.5%, by weight of the composition, the composition may provide hair volume down benefit.

Without intending to be limited by theory, it is believed that: the translucency of composition A of the present invention is due to a specific gel matrix provided by the cationic surfactant comprising: dialkyl dimethyl ammonium salts and the combination of amidoamines and acids, together with high melting point fatty compounds and aqueous carrier. Compositions made by other cationic surfactants such as (i) the single use of dialkyl dimethyl ammonium salts, (ii) the single use of the combination of amidoamines and acids, and (iii) the single use of monoalkyl trimethyl ammonium chloride, are known to be opaque. Without intending to be limited by theory, it is also believed that: the specific gel matrix of composition A is different in structure from gel matrixes providing opaque compositions. Without intending to be limited by theory, it is believed that: cellulose polymers also help provide a translucent composition.

In another preferred embodiment of the present invention, the composition_(Composition B) comprises by weight:

(a) from about 0.001% to about 5% of the cellulose polymer, preferably the cellulose polymer comprising a hydrophobically modified cationic cellulose;

(b) from about 0.01% to about 10% of a cationic surfactant comprising an amidoamine and an acid;

(c) from about 0.01% to about 15% of a high melting point fatty compound having a melting point of 25° C. or higher;

(d) an aqueous carrier; and

(e) from about 0.01% to about 10% of a silicone compound being a hydrophilic silicone.

Preferably composition B further comprises a polypropylene glycol. Preferably, in composition B, the cellulose polymer further comprises a hydrophilic cellulose polymer in addition to the hydrophobically modified cationic cellulose. Composition B can provide hair volume control benefit, especially hair volume-up benefit, in addition to other conditioning benefits. Furthermore, composition B can provide improved wet feel such as slippery and slick feel, and easiness to comb on wet hair.

Cellulose Polymer

The hair conditioning composition of the present invention comprises a cellulose polymer. The cellulose polymers useful herein provide hair volume control benefit while not deteriorating conditioning benefits such as fly-away control.

The cellulose polymers useful herein are those having a molecular weight of from about 10,000 to about 10,000,000, preferably from about 10,000 to about 5,000,000, more preferably from about 10,000 to about 2,000,000.

The cellulose polymers useful herein are included in the hair conditioning composition of the present invention at a level by weight of from about 0.001% to about 5%, preferably from about 0.01% to about 2.0%, more preferably from about 0.05% to about 1.0%.

The cellulose polymers useful herein can be cationic, nonionic, or anionic. The cellulose polymers useful herein can be hydrophobically modified or non-hydrophobically modified. Preferred cellulose polymers are cationic or nonionic, and/or hydrophobically modified.

The cellulose polymers useful herein include, for example, acacia gum, algin, alginic acid, arabia gum, carob gum, carrageenan, cellulose gum, cetyl hydroxyethyl cellulose, chondoitin sulfate, cocodimonium hydroxypropyloxyethylcellulose, ethyl cellulose, guar gum, guar hydroxytrimonium chloride, guar hydroxypropyltrimonium chloride, hydroxybutyl cellulose, hydroxyethyl cellulose, hydroxyethyl ethylcellulose, hydroxymethyl cellulose, hydroxypropyl cellulose, hydroxypropyl guar, hydroxypropyl guar hydroxypropyl trimonium chloride, hydroxypropyl methylcellulose, karaya gum, laurdimonium hydroxypropyl oxyethyl cellulose, locust bean gum, methyl cellulose, microcrystalline cellulose, nitro cellulose, nonoxynyl hydroxyethylcellulose, pectin, polyquaternium-4, polyquaternium-10, polyquaternium-24, quince seed gum (Cydonia oblonga Mill), sclerotium gum, sodium carboxymethyl cellulose, sodium cellulose sulfate, sodium hyaluronate, tragacanth gum, xanthan gum.

Other cellulose polymers useful herein include, for example, polysaccharide derivatives such as aluminum starch octenyl succinate, cornstarch, and distarch phosphate.

Preferably, the cellulose polymers are selected from the group consisting of: hydrophobically modified cationic celluloses such as Polyquaternium 24; hydrophobically modified cellulose ethers such as cetyl hydroxyethylcellulose; a copolymer of hydrophilic-cellulose units and diallyldimethyl ammonium chloride units such as Polyquaternium-4; a polymeric quaternary ammonium salt of hydroxyethyl cellulose reacted with a trimethyl ammonium substituted epoxide such as Polyquaternium-10; guar derivatives such as guar hydroxytrimonium chloride, guar hydroxypropyltrimonium chloride, hydroxypropyl guar, and hydroxypropyl guar hydroxypropyl trimonium chloride; and mixtures thereof.

Hydrophilic cellulose polymers are preferably used in composition B, together with the above preferred cellulose polymers. The hydrophilic cellulose useful herein includes, for example, hydroxymethyl cellulose, hydroxyethyl cellulose, hydroxypropyl cellulose, hydroxybuthyl cellulose, hydroxymethyl ethylcellulose, hydroxyethyl methylcellulose, hydroxyethyl ethylcellulose, hydroxypropyl ehtylcellulose, hydroxypropyl methylcellulose, and hydroxybutyl ethylcellulose.

Hydrophobically Modified Cationic Cellulose

The hydrophobically modified cationic celluloses useful in the present invention are those having the following formula:

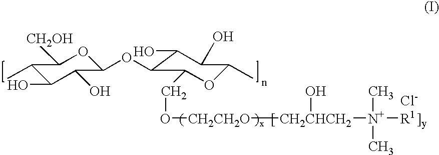

wherein R¹ is an alkyl having from about 8 to about 22 carbons, preferably from about 10 to about 18 carbons; n is an integer from 1 to about 35,000, preferably from about 100 to about 8,000; x is 0 or an integer from 1 to about 6, preferably from about 1 to about 3; and y is the level of cationic substitution from 0.1 to 1.0. The hydrophobically modified cationic celluloses useful herein must have a molecular weight of from about 50,000 to about 10,000,000, preferably from about 200,000 to about 2,000,000.

Commercially available hydrophobically modified cationic celluloses include, for example, the polymeric quaternary ammonium salts of hydroxyethyl cellulose reacted with lauryl dimethyl ammonium-substituted epoxide, referred to in the industry (CTFA) as Polyquaternium 24, available from Amerchol Corp. (Edison, N.J., USA) under the tradename Polymer LM-200®.

Hydrophobically Modified Cellulose Ether

The hydrophobically modified cellulose ethers useful herein comprise a hydrophilic cellulose backbone and a hydrophobic substitution group. The hydrophilic cellulose backbone has a sufficient degree of nonionic substitution to cellulose to be water soluble. Such hydrophilic cellulose backbone is selected from the group consisting of methyl cellulose, hydroxymethyl cellulose, hydroxyethyl cellulose, hydroxyethyl ethylcellulose, hydroxypropyl cellulose, hydroxypropyl methylcellulose, hydroxybutyl cellulose, and mixtures thereof. The amount of nonionic substitution is not critical, so long as there is an amount sufficient to assure that the hydrophilic cellulose backbone is water soluble. The hydrophilic cellulose backbone has a molecular weight of about less than 1,000,000, preferably from about 20,000 to about 700,000, or from about 75 D. P. to about 2500 D. P. Further, where a high viscosity building effect is not desirable, a lower molecular weight cellulose backbone is preferred. One of the preferred hydrophilic cellulose backbone is hydroxyethyl cellulose having a molecular weight of from about 50,000 to about 700,000. Hydroxyethyl cellulose of this molecular weight is known to be one of the most hydrophilic of the materials contemplated. Thus, hydroxyethyl cellulose can be modified to a greater extent than other hydrophilic cellulose backbones.

The hydrophilic cellulose backbone is further substituted with a hydrophobic substitution group via an ether linkage to render the hydrophobically modified cellulose ether to have less than 1% water solubility, preferably less than 0.2% water solubility. The hydrophobic substitution group is selected from a straight or branched chain alkyl group of from about 10 to about 22 carbons; wherein the ratio of the hydrophilic groups in the hydrophilic cellulose backbone to the hydrophobic substitution group being from about 2:1 to about 1000:1, preferably from about 10:1 to about 100:1.

Commercially available hydrophobically modified cellulose ethers useful herein include: cetyl hydroxyethylcellulose having tradenames NATROSOL PLUS 330CS and POLYSURF 67, both available from Aqualon Company, Del, USA, having cetyl group substitution of about 0.4% to about 0.65% by weight of the entire polymer.

Copolymer of Hydrophilic-Cellulose and Diallyldimethyl Ammonium Chloride

The copolymers of hydrophilic-cellulose and diallyldimethyl ammonium chloride useful herein are those having hydrophilic-cellulose units and diallyldimethyl ammonium chloride units, the hydrophilic cellulose units selected from the group consisting of methyl cellulose, hydroxymethyl cellulose, hydroxyethyl cellulose, hydroxyethyl ethylcellulose, hydroxypropyl cellulose, hydroxypropyl methylcellulose, hydroxybutyl cellulose, and mixtures thereof, preferably hydroxyethylcellulose. The ratio of the number of the hydrophilic-cellulose units to the diallyldimethyl ammonium chloride units comprised in the copolymer is from about 1:100 to about 10:1, preferably from about 1:10 to about 10:1, more preferably from about 1:3 to about 5:1, still preferably from about 1:1 to about 3:1, and wherein the molecular weight of the copolymer is from about 10,000 to about 250,000, preferably from about 15,000 to about 200,000. To provide preferred copolymers, the hydrophilic-cellulose units are contained at a range of from about 40 to about 350, and the diallyldimethyl ammonium chloride units are contained at a range of from about 40 to about 120.

Highly suitable copolymers are hydroxyethylcellulose diallyldimethyl ammonium chloride copolymers known in the industry as Polyquaternium-4 (CTFA Dictionary). Commercially available hydroxyethylcellulose diallyldimethyl ammonium chloride copolymers are those with tradenames CELQUAT L-200 and CELQUAT H-100 available from National Starch Corp.

Cationic Surfactant

The hair conditioning composition of the present invention comprises a cationic surfactant. The cationic surfactant, together with a high melting point fatty compound and an aqueous carrier, provide a gel network which is suitable for providing various conditioning benefits such as slippery and slick feel on wet hair, and softness, moisturized feel, and fly-away control on dry hair. Preferably the composition of the present invention is substantially free of certain components such as of anionic surfactants and anionic polymers, since the gel matrix may become unstable or, at worst, become destroyed in the presence of such components.

Among a variety of cationic surfactants described below, cationic surfactants comprising amidoamines and acids are preferably used in the composition of the present invention in view of providing conditioning benefits such as softness, moisturized feel, and fly-away control, as described above. Dialkyl dimethyl ammonium salts are also preferably used together with the cationic surfactants comprising amidoamines and acids, in view of providing translucency to the composition in addition to the conditioning benefits described above.

The cationic surfactant useful herein is any known to the artisan, and can be included in the composition at a level by weight of, preferably from about 0.01% to about 10%, more preferably from about 0.1% to about 8%, still more preferably from about 0.5 to about 5%, by weight of the composition.

Among the cationic surfactants useful herein are those corresponding to the general formula (I):

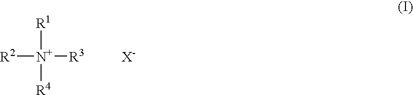

wherein at least one of R¹, R², R³, and R⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R¹, R², R³, and R⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is when R¹, R², R³, and R⁴ are independently selected from C₁ to about C₂₂ alkyl. Nonlimiting examples of cationic surfactants useful in the present invention include the materials having the following CTFA designations: quaternium-8, quaternium-14, quaternium-18, quaternium-18 methosulfate, quaternium-24, and mixtures thereof.

Among the cationic surfactants of general formula (I), preferred are those containing in the molecule at least one alkyl chain having at least 16 carbons. Nonlimiting examples of such preferred cationic surfactants include: behenyl trimethyl ammonium chloride, cetyl trimethyl ammonium chloride, hydrogenated tallow alkyl trimethyl ammonium chloride, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate) ammonium chloride, and N-(stearoyl colamino formyl methy) pyridinium chloride.

Among the cationic surfactants of general formula (I), dialkyl dimethyl ammonium salts are preferably contained in composition A of the present invention. Dialkyl dimethyl ammonium salts useful herein are those having two long alkyl chains having from 8 to 30 carbons, preferably from 12 to 22 carbons. The dialkyl dimethyl ammonium salts useful herein include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, and dibehenyl dimethyl ammonium chloride. Among them, preferred is distearyl dimethyl ammonium chloride.

Also preferred are hydrophilically substituted cationic surfactants in which at least one of the substituents contain one or more aromatic, ether, ester, amido, or amino moieties present as substituents or as linkages in the radical chain, wherein at least one of the R¹-R⁴ radicals contain one or more hydrophilic moieties selected from alkoxy (preferably C₁-C₃ alkoxy), polyoxyalkylene (preferably C₁-C₃ polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Preferred hydrophilically substituted cationic surfactants include those of the formula (II) through (VIII) below:

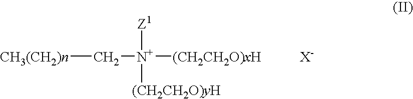

wherein n is from 8 to about 28, x+y is from 2 to about 40, Z¹ is a short chain alkyl, preferably a C₁-C₃ alkyl, more preferably methyl, or (CH₂CH₂O)_{z}H wherein x+y+z is up to 60, and X is a salt forming anion as defined above;

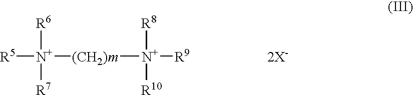

wherein m is 1 to 5, one or more of R⁵, R⁶, and R⁷ are independently an C₁-C₃₀ alkyl, the remainder are CH₂CH₂OH, one or two of R⁸, R⁹, and R¹⁰ are independently an C₁-C₃₀ alkyl, and remainder are CH₂CH₂OH, and X is a salt forming anion as mentioned above;

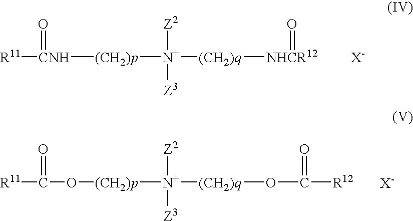

wherein, independently for formulae (IV) and (V), Z² is an alkyl, preferably a C₁-C₃ alkyl, more preferably methyl, and Z³ is a short chain hydroxyalkyl, preferably hydroxymethyl or hydroxyethyl, p and q independently are integers from 2 to 4, inclusive, preferably from 2 to 3, inclusive, more preferably 2, R¹¹ and R¹², independently, are substituted or unsubstituted hydrocarbyls, preferably C₁₂-C₂₀ alkyl or alkenyl, and X is a salt forming anion as defined above;

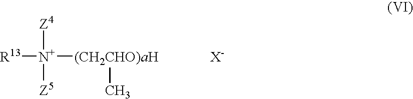

wherein R¹³ is a hydrocarbyl, preferably a C₁-C₃ alkyl, more preferably methyl, Z⁴ and Z⁵ are, independently, short chain hydrocarbyls, preferably C₂-C₄ alkyl or alkenyl, more preferably ethyl, a is from 2 to about 40, preferably from about 7 to about 30, and X is a salt forming anion as defined above;

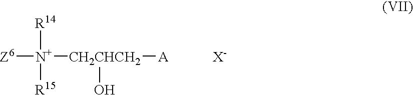

wherein R¹⁴ and R¹⁵, independently, are C₁-C₃ alkyl, preferably methyl, Z⁶ is a C₁₂-C₂₂ hydrocarbyl, alkyl carboxy or alkylamido, and A is a protein, preferably a collagen, keratin, milk protein, silk, soy protein, wheat protein, or hydrolyzed forms thereof; and X is a salt forming anion as defined above;

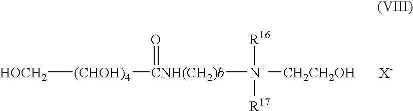

wherein b is 2 or 3, R¹⁶ and R¹⁷, independently are C₁-C₃ hydrocarbyls preferably methyl, and X is a salt forming anion as defined above. Nonlimiting examples of hydrophilically substituted cationic surfactants useful in the present invention include the materials having the following CTFA designations: quaternium-16, quaternium-26, quaternium-27, quaternium-30, quaternium-33, quaternium-43, quaternium-52, quaternium-53, quaternium-56, quaternium-60, quaternium-61, quaternium-62, quaternium-70, quaternium-71, quaternium-72, quaternium-75, quaternium-76 hydrolyzed collagen, quaternium-77, quaternium-78, quaternium-79 hydrolyzed collagen, quaternium-79 hydrolyzed keratin, quaternium-79 hydrolyzed milk protein, quaternium-79 hydrolyzed silk, quaternium-79 hydrolyzed soy protein, and quaternium-79 hydrolyzed wheat protein, quaternium-80, quaternium-81, quaternium-82, quaternium-83, quaternium-84, and mixtures thereof.

Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commerically available under the following tradenames; VARISOFT 110, VARIQUAT K1215 and 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas.

Salts of primary, secondary, and tertiary fatty amines are also suitable cationic surfactants. The alkyl groups of such amines preferably have from about 12 to about 22 carbon atoms, and can be substituted or unsubstituted. Particularly useful are amidoamines of the following general formula:

R¹CONH(CH₂)ₘN(R²)₂

wherein R¹ is a residue of C₁₁ to C₂₄ fatty acids, R² is a C₁ to C₄ alkyl, and m is an integer from 1 to 4.

Preferred amidoamine useful in the present invention includes stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, and mixtures thereof; more preferably stearamidopropyldimethylamine, stearamidoethyldiethylamine, and mixtures thereof.

The amidoamines herein are preferably used in combination with acids selected from the group consisting of L-glutamic acid, lactic acid, hydrochloric acid, malic acid, succinic acid, acetic acid, fumaric acid, L-glutamic acid hydrochloride, tartaric acid, citric acid, and mixtures thereof; preferably L-glutamic acid, lactic acid, hydrochloric acid, and mixtures thereof. Preferably, the mole ratio of amidoamine to acid is from about 1:0.3 to about 1:1, more preferably from about 1:0.5 to about 1:0.9. Such combinations of amidoamines and acids are preferably contained in the compositions of the present invention.

High Melting Point Fatty Compound

The hair conditioning composition of the present invention comprises a high melting point fatty compound. The high melting fatty compound, together with a cationic surfactant and an aqueous carrier, provide a gel matrix which is suitable for providing various conditioning benefits such as slippery and slick feel on wet hair, and softness, moisturized feel, and fly-away control on dry hair.

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

The high melting point fatty compound can be included in the composition at a level of, preferably from about 0.1% to about 15%, more preferably from about 0.5% to about 10%, still more preferably from about 1% to about 7%, by weight of the composition.

The fatty alcohols useful herein are those having from about 14 to about 30 carbon atoms, preferably from about 16 to about 22 carbon atoms. These fatty alcohols are saturated and can be straight or branched chain alcohols. Nonlimiting examples of fatty alcohols include, cetyl alcohol, stearyl alcohol, behenyl alcohol, and mixtures thereof.

The fatty acids useful herein are those having from about 10 to about 30 carbon atoms, preferably from about 12 to about 22 carbon atoms, and more preferably from about 16 to about 22 carbon atoms. These fatty acids are saturated and can be straight or branched chain acids. Also included are diacids, triacids, and other multiple acids which meet the requirements herein. Also included herein are salts of these fatty acids. Nonlimiting examples of fatty acids include lauric acid, palmitic acid, stearic acid, behenic acid, sebacic acid, and mixtures thereof.

The fatty alcohol derivatives and fatty acid derivatives useful herein include alkyl ethers of fatty alcohols, alkoxylated fatty alcohols, alkyl ethers of alkoxylated fatty alcohols, esters of fatty alcohols, fatty acid esters of compounds having esterifiable hydroxy groups, hydroxy-substituted fatty acids, and mixtures thereof. Nonlimiting examples of fatty alcohol derivatives and fatty acid derivatives include materials such as methyl stearyl ether; the ceteth series of compounds such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C₁-C₃₀ alkyl ethers of the ceteth, steareth, and ceteareth compounds just described; polyoxyethylene ethers of behenyl alcohol; ethyl stearate, cetyl stearate, cetyl palmitate, stearyl stearate, myristyl myristate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, glyceryl monostearate, glyceryl distearate, glyceryl tristearate, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

Commercially available high melting point fatty compounds useful herein include: cetyl alcohol, stearyl alcohol, and behenyl alcohol having tradenames KONOL series available from Shin Nihon Rika (Osaka, Japan), and NAA series available from NOF (Tokyo, Japan); pure behenyl alcohol having tradename 1-DOCOSANOL available from WAKO (Osaka, Japan), various fatty acids having tradenames NEO-FAT available from Akzo (Chicago Ill., USA), HYSTRENE available from Witco Corp. (Dublin Ohio, USA), and DERMA available from Vevy (Genova, Italy).

Aqueous Carrier

The hair conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 95%, preferably from about 30% to about 92%, and more preferably from about 50% to about 90% water.

Silicone Compound

The hair conditioning composition of the present invention preferably contains a silicone compound. The silicone compound can be included in the composition at a level by weight of, preferably from about 0.01% to about 10%, more preferably from about 0.05% to about 8%, still more preferably from about 0.1% to about 5%.

The silicone compounds hereof can include volatile soluble or insoluble, or nonvolatile soluble or insoluble silicone conditioning agents. By soluble what is meant is that the silicone compound is miscible with the carrier of the composition so as to form part of the same phase. By insoluble what is meant is that the silicone forms a separate, discontinuous phase from the carrier, such as in the form of an emulsion or a suspension of droplets of the silicone. The silicone compounds herein may be made by conventional polymerization, or emulsion polymerization.

The silicone compounds for use herein will preferably have a viscosity of from about 5 to about 2,000,000 centistokes at 250C, more preferably from about 100 to about 1,000,000, and even more preferably from about 500 to about 500,000. The viscosity can be measured by means of a glass capillary viscometer as set forth in Dow Corning Corporate Test Method CTM0004, Jul. 20, 1970, which is incorporated by reference herein in its entirety. Silicone compound of high molecular weight may be made by emulsion polymerization.

Silicone compounds useful herein include hydrophilic silicones, polyalkyl polyaryl siloxanes, polyalkyleneoxide-modified siloxanes, silicone resins, amino-substituted siloxanes, and mixtures thereof. The silicone compound is preferably selected from the group consisting of hydrophilic silicones, polyalkyl polyaryl siloxanes, polyalkyleneoxide-modified siloxanes, silicone resins, and mixtures thereof, and more preferably selected from the group consisting of hydrophilic silicones, polyalkyl polyaryl siloxanes, and mixtures thereof.

Hydrophilic silicones useful herein include those having the following formula (I):

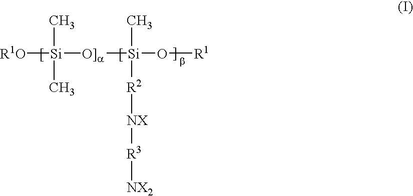

wherein R′ is an aliphatic group having 8-30 carbon atoms, preferably aliphatic group having 12-22 carbon atoms; α is an integer of from about 1 to about 200, preferably from about 20 to about 100; β is an integer of from about 1 to about 2; R² is an aliphatic group having 2-8 carbon atoms, preferably aliphatic group having 24 carbon atoms; R³ is an aliphatic group having 2-8 carbon atoms, preferably aliphatic group having 24 carbon atoms; X is a hydroxyaliphatic group having 2-8 carbon atoms and 1-5 hydroxyl groups, preferably hydroxyaliphatic group having 24 carbon atoms and 1-3 hydroxyl groups. This hydrophilic silicone can be a block copolymer as described above in the formula (I), or can be a random copolymer of the unit having the integer α and the unit having the integer 0. In case of random copolymer, total numbers of integers α and β are as those described above.

Commercially available hydrophilic silicones useful herein include, for example, Bis (C13-C15 Alkoxy) PG Amodimethicone available from Dow corning with tradename DC8600 as a mixture with PEG/PPG-24/24 Methyl Ether Glycidoxy Dimethicone.

Hydrophilic silicones useful herein also include those having the following formula (II):

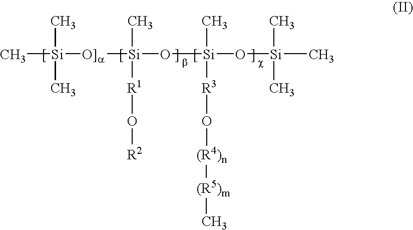

wherein α is an integer of from about 1 to about 300, preferably from about 20 to about 120, more preferably from about 20 to about 100; β is an integer of from about 1 to about 3; χ is an integer of from about 1 to about 3; R′ is an aliphatic group having 1-8 carbon atoms, preferably aliphatic group having 24 carbon atoms; R² is an epoxy group having an aliphatic substitution with 2-8 carbon atoms, preferably epoxy group having an aliphatic substitution with 24 carbon atoms; R³ is an aliphatic group having 1-8 carbon atoms, preferably aliphatic group having 24 carbon atoms; R⁴ is an alkoxy group having 1-8 carbon atoms, preferably alkoxy group having 2-4 carbon atoms; R⁵ is an alkoxy group having 1-8 carbon atoms, preferably alkoxy group having 24 carbon atoms; n is an integer of from 1 to 300; m is an integer of from 1 to 300. This hydrophilic silicone can be a block copolymer as described above in the formula (II), or can be a random copolymer of the unit having the integer β, the unit having the integer, and the unit having the integer χ. In case of random copolymer, total numbers of integers α, β, and χ are as those described above.

Commercially available hydrophilic silicones useful herein include, for example, PEG/PPG-24/24 Methyl Ether Glycidoxy Dimethicone available from Dow coming with tradename DC8600 as a mixture with Bis (C13-C15 Alkoxy) PG Amodimethicone.

Among these hydrophilic silicones, preferred is the hydrophilic silicone having the formula (I).

Polyalkyl polyaryl siloxanes useful here in include those with the following structure (I)

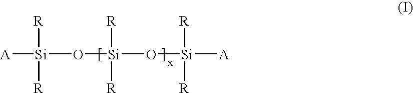

wherein R is alkyl or aryl, and x is an integer from about 7 to about 8,000. “A” represents groups which block the ends of the silicone chains. The alkyl or aryl groups substituted on the siloxane chain (R) or at the ends of the siloxane chains (A) can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable A groups include hydroxy, methyl, methoxy, ethoxy, propoxy, and aryloxy. The two R groups on the silicon atom may represent the same group or different groups. Preferably, the two R groups represent the same group. Suitable R groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their ViscasilR and SF 96 series, and from Dow Corning in their Dow Corning 200 series. Polymethylphenylsiloxanes, for example, from the General Electric Company as SF 1075 methyl phenyl fluid or from Dow Corning as 556 Cosmetic Grade Fluid, are useful herein.

Also preferred, for enhancing the shine characteristics of hair, are highly arylated silicone compounds, such as highly phenylated polyethyl silicone having refractive index of about 1.46 or higher, especially about 1.52 or higher. When these high refractive index silicone compounds are used, they should be mixed with a spreading agent, such as a surfactant or a silicone resin, as described below to decrease the surface tension and enhance the film forming ability of the material.

Another polyalkyl polyaryl siloxane that can be especially useful is a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. Silicone gums are described by Petrarch, and others including U.S. Pat. No. 4,152,416, to Spitzer et al., issued May 1, 1979 and Noll, Walter, Chemistry and Technology of Silicones, New York: Academic Press 1968. Also describing silicone gums are General Electric Silicone Rubber Product Data Sheets SE 30, SE 33, SE 54 and SE 76. All of these described references are incorporated herein by reference in their entirety. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof.

Polyalkyleneoxide-modified siloxanes useful herein include, for example, polypropylene oxide modified and polyethylene oxide modified polydimethylsiloxane. The ethylene oxide and polypropylene oxide level should be sufficiently low so as not to interfere with the dispersibility characteristics of the silicone. These materials are also known as dimethicone copolyols.

Silicone resins, which are highly crosslinked polymeric siloxane systems, are useful herein. The crosslinking is introduced through the incorporation of tri-functional and tetra-functional silanes with mono-functional or di-functional, or both, silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetrafunctional siloxane monomer units, and hence, a sufficient level of crosslinking, such that they dry down to a rigid, or hard, film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. Preferably, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinylchlorosilanes, and tetrachlorosilane, with the methyl substituted silanes being most commonly utilized. Preferred resins are offered by General Electric as GE SS4230 and SS4267. Commercially available silicone resins will generally be supplied in a dissolved form in a low viscosity volatile or nonvolatile silicone fluid. The silicone resins for use herein should be supplied and incorporated into the present compositions in such dissolved form, as will be readily apparent to those skilled in the art. Without being bound by theory, it is believed that the silicone resins can enhance deposition of other silicone compounds on the hair and can enhance the glossiness of hair with high refractive index volumes.

Other useful silicone resins are silicone resin powders such as the material given the CTFA designation polymethylsilsequioxane, which is commercially available as Tospearl™ from Toshiba Silicones.

Silicone resins can conveniently be identified according to a shorthand nomenclature system well known to those skilled in the art as the “MDTQ” nomenclature. Under this system, the silicone is described according to the presence of various siloxane monomer units which make up the silicone. Briefly, the symbol M denotes the mono-functional unit (CH₃)₃SiO)_{0.5}; D denotes the difunctional unit (CH₃)₂SiO; T denotes the trifunctional unit (CH₃)SiO_{1.5}; and Q denotes the quadri- or tetra-functional unit SiO₂. Primes of the unit symbols, e.g., M′, D′, T′, and Q′ denote substituents other than methyl, and must be specifically defined for each occurrence. Typical alternate substituents include groups such as vinyl, phenyl, amino, hydroxyl, etc. The molar ratios of the various units, either in terms of subscripts to the symbols indicating the total number of each type of unit in the silicone, or an average thereof, or as specifically indicated ratios in combination with molecular weight, complete the description of the silicone material under the MDTQ system. Higher relative molar amounts of T, Q, T′ and/or Q′ to D, D′, M and/or or M′ in a silicone resin is indicative of higher levels of crosslinking. As discussed before, however, the overall level of crosslinking can also be indicated by the oxygen to silicon ratio.

The silicone resins for use herein which are preferred are MQ, MT, MTQ, MQ and MDTQ resins. Thus, the preferred silicone substituent is methyl. Especially preferred are MQ resins wherein the M:Q ratio is from about 0.5:1.0 to about 1.5:1.0 and the average molecular weight of the resin is from about 1000 to about 10,000.

Amino-substituted siloxanes useful herein include those represented by the following structure (II)

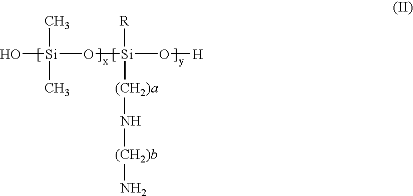

wherein R is CH₃ or OH, x and y are integers which depend on the molecular weight, the average molecular weight being approximately between 5,000 and 10,000. This polymer is also known as “amodimethicone”.

Suitable amino-substituted siloxane fluids include those represented by the formula (III)

(R₁)ₐG₃₋ₐ—Si—(—OSiG₂)ₙ—(—OSiG_{b}(R₁)_{2-b})ₘ—O—SiG₃₋ₐ(R₁)ₐ (II)

in which G is chosen from the group consisting of hydrogen, phenyl, OH, C₁-C₈ alkyl and preferably methyl; a denotes 0 or an integer from 1 to 3, and preferably equals 0; b denotes 0 or 1 and preferably equals 1; the sum n+m is a number from 1 to 2,000 and preferably from 50 to 150, n being able to denote a number from 0 to 1,999 and preferably from 49 to 149 and m being able to denote an integer from 1 to 2,000 and preferably from 1 to 10; R₁ is a monovalent radical of formula CqH_{2q}L in which q is an integer from 2 to 8 and L is chosen from the groups

—N(R₂)CH₂—CH₂—N(R₂)₂

—N(R₂)₂

—N(R₂)₃A⁻

—N(R₂)CH₂—CH₂—NR₂H₂A⁻

in which R₂ is chosen from the group consisting of hydrogen, phenyl, benzyl, a saturated hydrocarbon radical, preferably an alkyl radical containing from 1 to 20 carbon atoms, and A⁻ denotes a halide ion.

An especially preferred amino-substituted siloxane corresponding to formula (III) is the polymer known as “trimethylsilylamodimethicone”, of formula (IV):

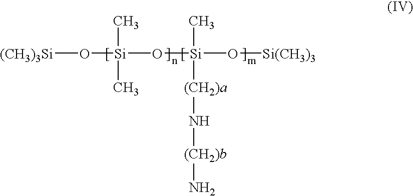

In this formula n and m are selected depending on the molecular weight of the compound desired.

Other amino-substituted siloxane which can be used are represented by the formula (V):

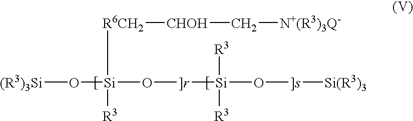

where R³ denotes a monovalent hydrocarbon radical having from 1 to 18 carbon atoms, preferably an alkyl or alkenyl radical such as methyl; R₄ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; r denotes an average statistical value from 2 to 20, preferably from 2 to 8; s denotes an average statistical value from 20 to 200, and preferably from 20 to 50. A preferred polymer of this class is available from Union Carbide under the name “UCAR SILICONE ALE 56.”

Polypropylene Glycol

The hair conditioning composition of the present invention preferably contains a polypropylene glycol having a weight average molecular weight of from about 200 g/mol to about 100,000 g/mol, preferably from about 1,000 g/mol to about 60,000 g/mol. Without intending to be limited by theory, it is believed that the polypropylene glycol herein deposits onto, or is absorbed into hair to act as a moisturizer buffer, and/or provides one or more other desirable hair conditioning benefits. As used herein, the term “polypropylene glycol” includes single-polypropylene glycol-chain segment polymers, and multi-polypropylene glycol-chain segment polymers. The general structure of branched polymers such as the multi-polypropylene glycol-chain segment polymers herein are described, for example, in “Principles of Polymerization,” pp. 17-19, G. Odian, (John Wiley & Sons, Inc., 3^{rd} ed., 1991).

The polypropylene glycols herein are typically polydisperse polymers. The polypropylene glycols useful herein have a polydispersity of from about 1 to about 2.5, preferably from about 1 to about 2, and more preferably from about 1 to about 1.5. As used herein, the term “polydispersity” indicates the degree of the molecular weight distribution of the polymer sample. Specifically, the polydispersity is a ratio, greater than 1, equal to the weight average molecular weight divided by the number average molecular weight. For a further discussion about polydispersity, see “Principles of Polymerization,” pp. 20-24, G. Odian, (John Wiley & Sons, Inc., 3^{rd} ed., 1991).

The polypropylene glycol useful herein may be either water-soluble, water-insoluble, or may have a limited solubility in water, depending upon the degree of polymerization and whether other moieties are attached thereto. The desired solubility of the polypropylene glycol in water will depend in large part upon the form (e.g., leave-on, or rinse-off form) of the hair care composition. The solubility in water of the polypropylene glycol herein may be chosen by the artisan according to a variety of factors. Accordingly, for a leave-on hair care composition, it is preferred that the polypropylene glycol herein be a water-soluble polypropylene glycol. Solubility information is readily available from polypropylene glycol suppliers, such as Sanyo Kasei (Osaka, Japan). However, the present invention may also take the form of a rinse-off hair care composition. Without intending to be limited by theory, it is believed that in such a composition, a water-soluble polypropylene glycol may be too easily washed away before it effectively deposits on hair and provides the desired benefit(s). For such a composition, a less soluble, or even a water-insoluble polypropylene glycol is therefore preferred. Accordingly, for a rinse-off hair care composition, it is preferred that the polypropylene glycol herein has a solubility in water at 25° C. of less than about 1 g/100 g water, more preferably a solubility in water of less than about 0.5 g/100 g water, and even more preferably a solubility in water of less than about 0.1 g/100 g water.

The polypropylene glycol can be included in the hair conditioning composition of the present invention at a level of, preferably from about 0.01% to about 10%, more preferably from about 0.05% to about 6%, still more preferably from about 0.1% to about 3% by weight of the composition.

Preferably the polypropylene glycol is selected from the group consisting of a single-polypropylene glycol-chain segment polymer, a multi-polypropylene glycol-chain segment polymer, and mixtures thereof, more preferably selected from the group consisting of a single-polypropylene glycol-chain segment polymer of Formula I, below, a multi-polypropylene glycol-chain segment polymer of Formula II, below, and mixtures thereof.

Single-Polypropylene Glycol-Chain Segment Polymer

Accordingly, a highly preferred single-polypropylene glycol-chain segment polymer has the formula:

HO—(C₃H₆O)ₐH (III),

wherein a is a value from about 4 to about 400, preferably from about 20 to about 100, and more preferably from about 20 to about 40.

The single-polypropylene glycol-chain segment polymer useful herein is typically inexpensive, and is readily available from, for example, Sanyo Kasei (Osaka, Japan), Dow Chemicals (Midland, Mich., USA), Calgon Chemical, Inc. (Skokie, Ill., USA), Arco Chemical Co. (Newton Square Pa., USA), Witco Chemicals Corp. (Greenwich, Conn., USA), and PPG Specialty Chemicals (Gurnee, Ill., USA).

Multi-Polypropylene Glycol-Chain Segment Polymer

A highly preferred multi-polypropylene glycol-chain segment polymer has the formula:

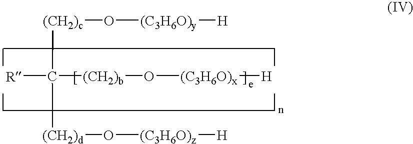

wherein n is a value from about 0 to about 10, preferably from about 0 to about 7, and more preferably from about 1 to about 4. In Formula IV, each R″ is independently selected from the group consisting of H, and C₁-C₃₀ alkyl, and preferably each R″ is independently selected from the group consisting of H, and C₁-C₄ alkyl. In Formula IV, each b is independently a value from about 0 to about 2, preferably from about 0 to about 1, and more preferably b=0. Similarly, c and d are independently a value from about 0 to about 2, preferably from about 0 to about 1. However, the total of b+c+d is at least about 2, preferably the total of b+c+d is from about 2 to about 3. Each e is independently a value of 0 or 1, if n is from about 1 to about 4, then e is preferably equal to 1. Also in Formula IV, x, y, and z is independently a value of from about 1 to about 120, preferably from about 7 to about 100, and more preferably from about 7 to about 100, where x+y+z is greater than about 20.

Examples of the multi-polypropylene glycol-chain segment polymer of Formula IV which is especially useful herein includes polyoxypropylene glyceryl ether (n=1, R′=H, b=0, c and d=1, e=1, and x, y, and z independently indicate the degree of polymerization of their respective polypropylene glycol-chain segments; available as New Pol GP-4000, from Sanyo Kasei, Osaka, Japan), polypropylene trimethylol propane (n=1, R′=C₂H₅, b=1, c and d=1, e=1, and x, y, and z independently indicate the degree of polymerization of their respective polypropylene glycol-chain segments), polyoxypropylene sorbitol (n=4, each R′=H, b=0, c and d=1, each e=1, and y, z, and each x independently indicate the degree of polymerization of their respective polypropylene glycol-chain segments; available as New Pol SP-4000, from Sanyo Kasei, Osaka, Japan), and PPG-10 butanediol (n=0, c and d=2, and y+z=10; available as Probutyl DB-10, from Croda, Inc., of Parsippany, N.J., U.S.A.).

In a preferred embodiment, one or more of the propylene repeating groups in the polypropylene glycol is an isopropyl oxide repeating group. More preferably one or more of the propylene oxide repeating groups of the polypropylene glycol of Formula III and/or the polypropylene glycol of Formula IV is an isopropyl oxide repeating group. Even more preferably, substantially all of the propylene oxide repeating groups of the polypropylene glycol of Formula III and/or the polypropylene glycol of Formula IV are isopropyl oxide repeating groups. Accordingly, a highly preferred single-polypropylene glycol-chain segment polymer has the formula:

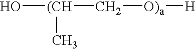

wherein a is defined as described above for Formula III. Similarly, a highly preferred multi-polypropylene glycol-chain segment polymer has the formula:

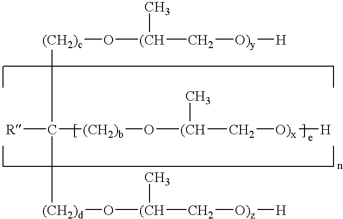

wherein n, R″, b, c, d, e, x, y, and z are defined as above, for Formula IV. It is recognized that the isopropyl oxide repeating groups may also correspond either alone, or in combination with the above depicted, to:

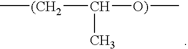

Aesthetic Material

The hair conditioning compositions of the present invention may include aesthetic materials. The aesthetic materials useful herein are those providing a benefit of pealized, macreous, and/or marbleized looks to the compositions of the present invention. The aesthetic materials useful herein include, for example, mica, titanium dioxide, bismuth oxychloride, aluminum oxide, chromium oxide, tin oxide, ferric ferrocyanide, carmine, silica, iron oxides, and mixtures thereof. The aesthetic materials can be included in the compositions of the present invention at a level by weight of, preferably from about 0.001% to about 2%, more preferably from about 0.01% to about 1%.

Commercially aesthetic materials include, for example, mixture of mica and titanium deoxide having tradename Flamenco super pearl available from Engelhard Corp., mixture of mica, titanium dioxide, and tin oxide having tradename Timiron available from Rona.

Vinylpyrrolidone Polymer

The hair conditioning composition of the present invention may further contain a vinylpyrrolidone polymer including vinylpyrrolidone copolymers and vinylpyrrolidone homopolymers such as polyvinylpyrrolidone and butylated polyvinylpyrrolidone, in view of providing increase in bulk hair volume. The vinylpyrrolidone copolymers useful herein are those which comprise monomers other than vinylpyrrolidone. The vinylpyrrolidone copolymers useful herein are believed to provide increase in bulk hair volume while not deteriorating conditioning benefits such as fly-away control.

Non-limiting examples of vinylpyrrolidone copolymers useful herein include polyvinylpyrrolidone/acrylates/lauryl methacrylate copolymer, polyvinylpyrrolidone/dimethiconylacrylate/polycarbamyl/polyglycol ester, polyvinylpyrrolidone/dimethylaminoethylmethacrylate copolymer, polyvinylpyrrolidone/dimethylaminoethylmethacrylate/polycarbamylpolyglycol ester, polyvinylpyrrolidone/DMAPA acrylates copolymer, polyvinylpyrrolidone/eicosene copolymer, polyvinylpyrrolidone/hexadecene copolymer, polyvinylpyrrolidone/polycarbamyl polyglycol ester, polyvinylpyrrolidone/vinyl acetate copolymer, polyvinylpyrrolidone/vinyl acetate/itaconic acid copolymer, polyvinylpyrrolidone/vinyl acetate/vinyl propionate copolymer, and polyvinylpyrrolidone/vinyl caprolactam/DMAPA acrylates copolymer, quaternized polyvinylpyrrolidone/dimethylaminoethylmethacrylate copolymer such as Polyquaternium-11, quaternized polyvinylpyrrolidone/methylvinylimidazolium chloride copolymer such as Polyquaternium-16, PVP/Vinyl Caprolactam/Dimethylaminoethyl Mathacrylate Copolymer, Polyquaternium-44, Polyquaternium-46, Polyquaternium-28, and Styrene/PVP Copolymer.

The copolymer of vinylpyrrolidone useful herein is preferably a water-soluble copolymer in view of better compatibility with gel matrix. The copolymer of vinylpyrrolidone useful herein is preferably a cationic copolymer or a nonionic copolymer in view of better compatibility with gel matrix and less deteriorating conditioning benefits.

Preferred vinylpyrrolidone copolymers are polyvinylpyrrolidone/dimethylaminoethylmethacrylate copolymer, quaternized polyvinylpyrrolidone/dimethylaminoethylmethacrylate copolymer such as Polyquaternium-11, quaternized polyvinylpyrrolidone/methylvinylimidazolium chloride copolymer such as Polyquaternium-16, and polyvinylpyrrolidone/vinyl acetate copolymer, in view of better compatibility with the gel matrix, less deteriorating conditioning benefits, and volume-up benefit. More preferred are polyvinylpyrrolidone/dimethylaminoethylmethacrylate copolymer and quaternized polyvinylpyrrolidone/dimethylaminoethylmethacrylate copolymer such as Polyquaternium-11.

Commercially available vinylpyrrolidone copolymers useful herein include: CTFA name polyvinylpyrrolidone/vinyl acetate copolymer having tradenames Luviskol VA28E, Luviskol VA37E, Luviskol VA55E, Luviskol VA64E, Luviskol VA73E, Luviskol VA37HM, Luviskol VA64 Powder, Luviskol VA64W, and Luviskol VA73W, all available from BASF, and PVP/VA E series, I series, S-630, all available from ISP; CTFA name polyvinylpyrrolidone/vinyl acetate/vinyl propionate copolymer having tradename Luviskol VAP343E available from BASF; CTFA name polyvinylpyrrolidone/acrylates/lauryl methacrylate copolymer having tradename Acrylidone LM available from ISP; CTFA name polyvinylpyrrolidone/dimethiconylacrylate/polycarbamyl/polyglycol ester having tradename Pecogel S-1120 available from Phoenix; CTFA name polyvinylpyrrolidone/dimethylaminoethylmethacrylate copolymer having tradename Copolymer 845, Copolymer 937, and Copolymer 958, all available from ISP; CTFA name polyvinylpyrrolidone/dimethylaminoethylmethacrylate/polycarbamylpolyglycol ester having tradename Pecogel GC-310 and Pecogel GC-1110 available from Phoenix; CTFA name polyvinylpyrrolidone/DMAPA acrylates copolymer having tradename ACP-1163 available from ISP; CTFA name polyvinylpyrrolidone/eicosene copolymer having tradename Antaron V-220 and Ganex V-220 available from ISP; CTFA name polyvinylpyrrolidone/hexadecene copolymer having tradename Antaron V-216 and Ganex V-216 available from ISP; CTFA name polyvinylpyrrolidone/polycarbamyl polyglycol ester having tradename Pecogel A-12 and Pecogel H series available from Phoenix; CTFA name polyvinylpyrrolidone/vinyl caprolactam/DMAPA acrylates copolymer having tradename ACP-1189 available from ISP; CTFA name Polyquaternium-11 having tradename Gafquat 734 and Gafquat 755N available from ISP; CTFA name Polyquaternium-16 having tradename Luviquat FC370 available from BASF; CTFA name PVP/DMAPA Acrylates Copolymer having tradename Styleze CC-10 available from ISP; CTFA name Polyquaternium-44 having tradename Luviquat Care and Luviquat MS-370 available from BASF; CTFA name Polyquaternium-46 having tradename Luviquat Hold available from BASF; CTFA name Vinyl Caprolactam/PVP/Dimethylaminoethyl Mathacrylate Copolymer having trade name Gaffix VC-713 available from ISP; CTFA name Polyquaternium-28 having tradename Gafquat HS-100 available from ISP; CTFA name Styrene/PVP Copolymer having tradename Polectron 430 and Antara 430 available from ISP.

The vinylpyrrolidone polymer can be included in the composition at a level by weight of, preferably from about 0.01% to about 10%, more preferably from about 0.05% to about 5%, still more preferably from about 0.1% to about 3%.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, a mixture of Polysorbate 60 and Cetearyl Alcohol with tradename Polawax NF available from Croda Chemicals, glycerylmonostearate available from Stepan Chemicals, hydroxyethyl cellulose available from Aqualon, hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Inositol, Sodium Benzoate, and Lactic acid) available from Goldschmidt, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate, antidandruff agents such as zinc pyridinethione; and optical brighteners, for example polystyrylstilbenes, triazinstilbenes, hydroxycoumarins, aminocoumarins, triazoles, pyrazolines, oxazoles, pyrenes, porphyrins, imidazoles, and mixtures thereof.

Polyethylene glycol

Polyethylene glycol can also be used as an additional component. The polyethylene glycols useful herein that are especially preferred are PEG-2M wherein n has an average value of about 2,000 (PEG-2M is also known as Polyox WSR® N-10 from Union Carbide and as PEG-2,000); PEG-SM wherein n has an average value of about 5,000 (PEG-SM is also known as Polyox WSR® N-35 and as Polyox WSR® N-80, both from Union Carbide and as PEG-5,000 and Polyethylene Glycol 300,000); PEG-7M wherein n has an average value of about 7,000 (PEG-7M is also known as Polyox WSR® N-750 from Union Carbide); PEG-9M wherein n has an average value of about 9,000 (PEG-9M is also known as Polyox WSR® N-3333 from Union Carbide); and PEG-14M wherein n has an average value of about 14,000 (PEG-14M is also known as Polyox WSR® N-3000 from Union Carbide).

Low melting point oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof.

Preferred low melting point oils herein are selected from the group consisting of: ester oils such as pentaerythritol ester oils, trimethylol ester oils, citrate ester oils, and glyceryl ester oils; poly α-olefin oils; and mixtures thereof, Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Such compounds are available from Kokyo Alcohol with tradenames KAKPTI, KAKTTI, and Shin-nihon Rika with tradenames PTO, ENUJERUBU TP3SO.

Particularly useful citrate ester oils herein include triisocetyl citrate with tradename CITMOL 316 available from Bernel, triisostearyl citrate with tradename PELEMOL TISC available from Phoenix, and trioctyldodecyl citrate with tradename CITMOL 320 available from Bemel.

Particularly useful glyceryl ester oils herein include triisostearin with tradename SUN ESPOL G-318 available from Taiyo Kagaku, triolein with tradename CITHROL GTO available from Croda Surfactants Ltd., trilinolein with tradename EFADERMA-F available from Vevy, or tradename EFA-GLYCERIDES from Brooks.

Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Mobil Chemical Co.

Cationic Polymer

The cationic polymers herein are other polymers than those described above under the titles “CELLULOSE POLYMER” and “VINYLPYRROLIDONE POLYMER”.

Preferably, the cationic polymer is a water-soluble cationic polymer. The cationic polymers hereof generally have an average molecular weight of at least about 5,000, typically from about 10,000 to about 10 million, preferably from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. The alkyl and dialkyl substituted monomers preferably have C₁-C₇ alkyl groups, more preferably C₁-C₃ alkyl groups. Other suitable spacer monomers include vinyl esters, vinyl alcohol (made by hydrolysis of polyvinyl acetate), maleic anhydride, propylene glycol, and ethylene glycol.

Suitable cationic hair conditioning polymers include, for example: cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, respectively; and mineral acid salts of amino-alkyl esters of homo- and co-polymers of unsaturated carboxylic acids having from 3 to 5 carbon atoms, as described in U.S. Pat. No. 4,009,256, incorporated herein by reference.

Product Forms

The hair conditioning compositions of the present invention can be in the form of rinse-off products or leave-on products, can be transparent or opaque, and can be formulated in a wide variety of product forms, including but not limited to creams, gels, emulsions, mousses and sprays.

In one preferred embodiment, the hair conditioning composition of the present invention is a rinse-off product.

In one preferred embodiment, the hair conditioning composition of the present invention can be translucent. What is meant by translucent, is that the composition has a L-value of from 0 to 60, preferably from 20 to 55, more preferably from 30 to 55.

The measurement of the L-value is done by a Macbeth Color Meter 1500/PLUS Color Measurement system (supplied by Kollmorgen Instruments Corporation), by using a transparent polystyrene cell having a thickness of 10 mm. The hair conditioning composition is put into the cell, and then, the cell is set in an appropriate position in the Macbeth 1500/PLUS Color Measurement system (Reflective Color Meter) for measurement. The measurement is done by using a light having a wave length of from about 340 nm to about 900 nm. Each measurement is repeated at least three times, and the average is obtained.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

The compositions of the present invention are suitable for rinse-off products and leave-on products, and are particularly useful for making products in the form of emulsion, cream, gel, spray or, mousse.

[Conditioner]

| Components | Cond. 1 | Cond. 2 | Cond. 3 | Cond. 4 | Cond. 5 |
|---|---|---|---|---|---|
| Hydrophobically modified cationic | 0.5 | — | — | — | — |
| cellulose *1 | | | | | |
| Hydrophobically modified cellulose | — | 0.25 | — | 0.5 | 0.5 |
| ether *2 | | | | | |
| Polyquaternium-4 *3 | — | — | 0.5 | — | — |
| Stearamidopropyl dimethylamine *4 | 1.2 | 1.2 | 1.2 | 1.2 | 1.2 |
| l-Glutamic acid *5 | 0.3 | 0.3 | 0.3 | 0.3 | 0.3 |
| Distearyl dimethyl ammonium | 1.0 | 1.0 | 1.0 | 1.0 | 1.0 |
| chloride *6 | | | | | |
| Cetyl alcohol *7 | 1.5 | 1.5 | 1.5 | 1.5 | 1.5 |
| Stearyl alcohol *8 | 2.7 | 2.7 | 2.7 | 2.7 | 2.7 |
| Hydrophilic silicone *9 | — | — | — | — | 0.4 |
| Polypropylene glycol *10 | 0.25 | 0.25 | 0.25 | 0.25 | 0.25 |
| Aesthetic material *11 | 0.1 | 0.1 | 0.1 | 0.1 | 0.1 |
| Polyvinylpyrrolidone/Dimethyl | — | — | — | 0.5 | 0.5 |
| aminoethylmethacrylate copolymer | | | | | |
| *12 | | | | | |
| PPG-3 Myristyl ether *18 | — | — | 0.25 | — | — |
| PPG-11 Stearyl ether *19 | — | — | — | 0.25 | — |
| Ethyl hexyl palmitate *20 | — | — | — | — | 0.5 |
| Preservatives | 0.033 | 0.033 | 0.033 | 0.033 | 0.033 |
| Benzyl alcohol | 0.4 | 0.4 | 0.4 | 0.4 | 0.4 |
| Perfume | 0.6 | 0.6 | 0.6 | 0.6 | 0.6 |
| Hydrolyzed collagen *13 | 0.01 | 0.01 | 0.01 | 0.01 | 0.01 |
| Vitamin E *14 | 0.01 | 0.01 | 0.01 | 0.01 | 0.01 |
| Panthenol *15 | 0.05 | 0.05 | 0.05 | 0.05 | 0.05 |
| Panthenyl ethyl ether *16 | 0.05 | 0.05 | 0.05 | 0.05 | 0.05 |
| Octyl methoxycinnamate | 0.09 | 0.09 | 0.09 | 0.09 | 0.09 |
| Benzophenone-3 | 0.09 | 0.09 | 0.09 | 0.09 | 0.09 |

| | |
|---|---|
| Citric Acid | amount necessary to adjust pH 3-7 |
| Deionized Water | q.s. to 100% |

| Components | Cond. 6 | Cond. 7 | Cond. 8 |
|---|---|---|---|
| Hydrophobically modified cationic | — | 0.5 | 0.5 |
| cellulose *1 | | | |
| Hydrophobically modified cellulose | 0.15 | — | — |
| ether *2 | | | |
| Polyquaternium-4 *3 | — | — | — |
| Stearamidopropyl dimethylamine *4 | 1.3 | 2.0 | 1.2 |
| l-Glutamic acid *5 | 0.3 | 0.6 | 0.4 |
| Distearyl dimethyl ammonium | 4.0 | 3.5 | — |
| chloride *6 | | | |
| Cetyl alcohol *7 | 1.5 | 1.5 | 1.5 |
| Stearyl alcohol *8 | 2.7 | 2.7 | 2.7 |
| Hydrophilic silicone *9 | — | 0.4 | 0.4 |
| Polypropylene glycol *10 | 0.25 | 0.25 | 0.25 |
| Aesthetic material *11 | 0.1 | 0.1 | — |
| Hydroxyethyl ethylcellulose *17 | — | — | 0.3 |
| PPG-3 Myristyl ether *18 | 1.0 | — | 0.25 |
| Preservatives | 0.033 | 0.033 | 0.033 |
| Benzyl alcohol | 0.4 | 0.4 | 0.4 |
| Perfume | 0.6 | 0.6 | 0.6 |
| Hydrolyzed collagen *13 | 0.01 | 0.01 | 0.01 |
| Vitamin E *14 | 0.01 | 0.01 | 0.01 |
| Panthenol *15 | 0.05 | 0.05 | 0.05 |
| Panthenyl ethyl ether *16 | 0.05 | 0.05 | 0.05 |
| Octyl methoxycinnamate | 0.09 | 0.09 | 0.09 |
| Benzophenone-3 | 0.09 | 0.09 | 0.09 |

| | |
|---|---|
| Citric Acid | amount necessary to adjust pH 3-7 |
| Deionized Water | q.s. to 100% |

Definitions of Components

1 Hydrophobically modified cationic cellulose: Quaterisoft Polymer LM 200 available from Amerchol, which has the following formula:

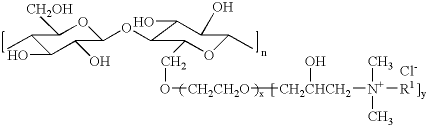

n=about 1,000

x=25-300

y=0.4

molecular weight=250,000-300,000

2 Hydrophobically modified cellulose ether: Cetyl hydroxyethylcellulose having tradename Polysurf 67 available from Aqualon.

3 Polyquaternium-4: Celquat L-200 available from National Starch Corp.

4 Stearamidopropyl dimethylamine: Amidoamine MPS available from Nikko.

5 l-Glutamic acid: l-Glutamic acid (cosmetic grade) available from Ajinomoto.

6 Distearyl dimethyl ammonium chloride: Available from Witco Chemicals

7 Cetyl alcohol: Konol series available from Shin Nihon Rika.

8 Stearyl alcohol: Konol series available from Shin Nihon Rika.

9 Hydrophilic silicone: mixture of Bis (C13-C15 Alkoxy) PG Amodimethicone and PEG/PPG-24/24 Methyl Ether Glycidoxy Dimethicone with tradename DC8600 available from Dow corning, wherein the Bis (C13-C15 Alkoxy) PG Amodimethicone has the following formula:

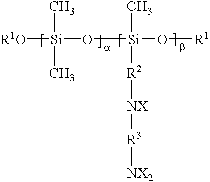

wherein R¹ is an alkyl group having 13-15 carbon atoms; α=20-100; β=1-2; R² is a branched alkyl group having the following formula: —CH₂CH(CH₃)CH₂—; R³ is an alkyl group having 2 carbon atoms; X is a hydroxyalkyl group having the formula:

CH₂CH₂CH(OH)CH₂OH;

and the PEG/PPG-24/24 Methyl Ether Glycidoxy Dimethicone has the following formula:

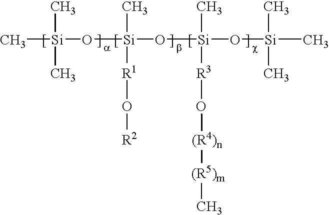

wherein α=20-100; β=1-2; χ=1-2; R¹ is an alkyl group having 3 carbon atoms; R² is an epoxy group having 2 carbon atoms; R³ is an alkyl group having 3 carbon atoms; R⁴ is an alkoxy group having 3 carbon atoms; R⁵ is an alkoxy group having 2 carbon atoms; n=1; m=1.

10 Polypropylene glycol: New pol PP-2000 available from Sanyo Kasei.

11 Aesthetic material: mixture of mica and titanium dioxide having tradename Flamenco super pearl available from Engelhard Corp.

12 Polyvinylpyrrolidone/Dimethylamino ethylmethacrylate copolymer: Copolymer 937 available from ISP.

13 Hydrolyzed collagen: Peptein 2000 available from Hormel.

14 Vitamin E: Emix-d available from Eisai.

15 Panthenol: available from Roche.

16 Panthenyl ethyl ether: available from Roche.

17 Hydroxyethyl ethylcellulose: Elfacos CD 481 available from Akzo Nobel

18 PPG-3 Myristyl ether: Varonic APM available from Goldschmidt

19 PPG-11 Stearyl ether: Varonic APS available from Goldschmidt

20 Ethyl hexyl palmitate: Tegosoft OP available from Goldschmidt

Method of Preparation

The hair conditioning compositions of “Cond. 1” through “Cond. 8” as shown above can be prepared by any conventional method well known in the art. They are suitably made as follows: Water is heated up to 80-85° C., and then, cellulose polymers and, if included, other polymeric materials can be dispersed in the water. Cationic surfactants such as amidoamine and acid, and dialkyl dimethyl ammonium salts are added in the solution with agitation. High melting point fatty compounds and preservatives are also added in the solution with agitation. The mixture thus obtained is cooled down to about 50° C. Cellulose polymers and, if included, other polymeric materials can be added to the mixture at this point with agitation, and, if present, the remaining components such as silicone compounds and polypropylene glycol are also added to the mixture with agitation. The mixture is further cooled down to about 30° C. Then, if included, aesthetic materials are added with agitation.

A triblender and/or mill can be used in each step, if necessary to disperse the materials.

The embodiments disclosed and represented by the previous “Cond. 1” through “Cond. 8” have many advantages. For example, they can provide conditioning benefits such as softness, moisturized feel, and fly-away control. They can also provide volume control benefit, “Cond. 1” through “Cond.5” and “Cond.8” can provide increase in bulk hair volume, and “Cond.6” and “Cond.7” can provide improved moisturizing benefit to achieve hair volume down. Furthermore, “Cond.1” through “Cond.3” and “Cond.6” and “Cond.7” can provide translucent composition.

Furthermore, “Cond. 4” through “Cond.6” and “Cond.8” can provide improved wet feel such as slippery and slick feel, and easiness to comb on wet hair.

It is understood that the examples and embodiments described herein are for illustrative purposes only and that various modifications or changes in light thereof will be suggested to one skilled in the art without departing from its spirit and scope.

What is claimed is:

1. A hair conditioning composition comprising by weight:
(a) from about 0.001% to about 5% of a cellulose polymer having a molecular weight of from about 10,000 to about 10,000,000;
(b) from about 0.01% to about 10% of a cationic surfactant;
(c) from about 0.01% to about 15% of a high melting point fatty compound having a melting point of 25° C. or higher; and
(d) an aqueous carrier.

2. The hair conditioning composition according to claim 1, wherein the cellulose polymer is selected from the group consisting of:
(i) a hydrophobically modified cellulose ether comprising a hydrophilic cellulose backbone and a hydrophobic substitution group; the hydrophilic cellulose backbone being water soluble and selected from the group consisting of methyl cellulose, hydroxymethyl cellulose, hydroxyethyl cellulose, hydroxyethyl ethylcellulose, hydroxypropyl cellulose, hydroxypropyl methylcellulose, hydroxybutyl cellulose, and mixtures thereof; and having grafted thereto the hydrophobic substitution group to render the hydrophobically modified cellulose ether to have less than 1% water solubility, the hydrophobic substitution group selected from a straight or branched chain alkyl group of from about 10 to about 22 carbons; wherein the ratio of the hydrophilic groups in the hydrophilic cellulose backbone to the hydrophobic substitution group being from about 2:1 to about 1000:1;
(ii) a hydrophobically modified cationic cellulose having the following formula: 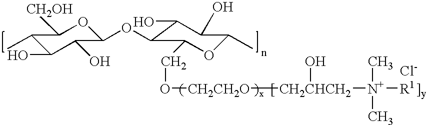
wherein R¹ is an alkyl having from about 8 to about 22 carbons, n is an integer from 1 to about 35,000; x is 0 or an integer from 1 to about 6; y is the level of cationic substitution from 0.1 to 1.0; and having a molecular weight of from about 50,000 to about 10,000,000;
(iii) a copolymer of hydrophilic-cellulose units and diallyldimethyl ammonium chloride units wherein the ratio of the number of hydrophilic-cellulose units to the diallyldimethyl ammonium chloride units is from about 1:100 to about 10:1, and wherein the molecular weight of the copolymer is from about 10,000 to about 250,000;
(iv) Polyquaternium-10;
(v) a guar derivative; and
(vi) mixtures thereof.

3. The hair conditioning composition according to claim 1, wherein the cationic surfactant comprises:
an amidoamine having the following general formula:
R¹CONH(CH₂)ₘN(R²)₂
wherein R¹ is a residue of C₁₁ to C₂₄ fatty acids, R² is a C₁ to C₄ alkyl, and m is an integer from 1 to 4; and
an acid selected from the group consisting of L-glutamic acid, lactic acid, hydrochloric acid, malic acid, succinic acid, acetic acid, fumaric acid, L-glutamic acid hydrochloride, tartaric acid, citric acid, and mixtures thereof.

4. The hair conditioning composition according to claim 3, wherein the conditioning agent further comprises a dialkyl dimethyl ammonium salt.

5. The hair conditioning composition according to claim 4 further comprising from about 0.001% to about 2% by weight of an aesthetic material.

6. The hair conditioning composition according to claim 1 further comprising from about 0.01% to about 10% of a silicone compound.

7. The hair conditioning composition according to claim 6, wherein the silicone compound comprises a hydrophilic silicone.

8. The hair conditioning composition according to claim 1 further comprises from about 0.01% to about 10% of a polypropylene glycol.

9. The hair conditioning composition according to claim 1 further comprises from about 0.01% to about 10% of a vinylpyrrolidone polymer.
